(19) United States
(12) Patent Application Publication
(10) Pub. No.: US 2026/0131153 A1
(43) Pub. Date: May 14, 2026

(54) WIRELESS ECOSYSTEM FOR A MEDICAL DEVICE

(71) Applicant: Cochlear Limited, Macquarie University, NSW (AU)

(72) Inventors: Jowan PITTEVILS, Bonheiden (BE); Werner MESKENS, Opwijk (BE)

(21) Appl. No.: 19/111,511

(22) PCT Filed: Sep. 1, 2023
(86) PCT No.: PCT/IB2023/058685

§ 371 Date: Mar. 13, 2025

Related U.S. Application Data
(60) Provisional application No. 63/408,611, filed on Sep. 21, 2022.

(51) Int. Cl. A61N 1/372; A61N 1/36; H04L 67/12; H04W 76/20; H04W 80/02

(52) U.S. Cl. CPC A61N 1/3727; A61N 1/36038; H04L 67/12; H04W 76/20; H04W 80/02

(57) ABSTRACT

Presented herein are techniques for adjusting one or more parameters or operations associated with a first wireless link operating in accordance with a first wireless protocol (e.g., a non-standardized/proprietary wireless protocol) based on one or more parameters or operations associated with a second wireless link operating in accordance with a second wireless protocol (e.g., a standardized protocol).

### FIELD OF THE INVENTION

The present invention relates generally to adjusting operations of a non-standardized wireless protocol associated with a medical device.

### RELATED ART

Medical devices have provided a wide range of therapeutic benefits to recipients over recent decades. Medical devices can include internal or implantable components/devices, external or wearable components/devices, or combinations thereof (e.g., a device having an external component communicating with an implantable component). Medical devices, such as traditional hearing aids, partially or fully-implantable hearing prostheses (e.g., bone conduction devices, mechanical stimulators, cochlear implants, etc.), pacemakers, defibrillators, functional electrical stimulation devices, and other medical devices, have been successful in performing lifesaving and/or lifestyle enhancement functions and/or recipient monitoring for a number of years.

The types of medical devices and the ranges of functions performed thereby have increased over the years. For example, many medical devices, sometimes referred to as “implantable medical devices,” now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, implantable components.

### SUMMARY

In one aspect, a method is provided. The method comprises: transmitting, from an external portion of a hearing device to an implantable portion of the hearing device, first wireless data over a first wireless link operating in accordance with a first wireless protocol; receiving second wireless data from an external device over a second wireless link operating in accordance with a second wireless protocol; and adjusting operations associated with the first wireless protocol based on the operation of the second wireless protocol.

In another aspect, one or more non-transitory computer readable storage media are provided. The one or more non-transitory computer readable storage media comprise instructions that, when executed by a processor, cause the processor to: transmit first data packets from an external portion of a medical device to an implantable portion of the medical device a first wireless link; receive second data packets from an external device over a second wireless link; and adjust one or more operating parameters of the first wireless link based on one or more operating parameters associated with the second wireless link.

In another aspect, a device is provided. The device comprises: at least one wireless interface; a memory; and at least one processor configured to: transmit, via the at least one wireless interface, first wireless data in accordance with a first wireless protocol, receive, via the at least one wireless interface, second wireless data sent in accordance with a second wireless protocol, and adjust one or more parameters of the first wireless protocol data based on one or more parameters of the second wireless protocol.

### BRIEF DESCRIPTION OF THE DRAWINGS

Embodiments of the present invention are described herein in conjunction with the accompanying drawings, in which:

FIG. 1A is a schematic diagram illustrating a cochlear implant system with which aspects of the techniques presented herein can be implemented;

FIG. 1B is a side view of a recipient wearing a sound processing unit of the cochlear implant system of FIG. 1A;

FIG. 1C is a schematic view of components of the cochlear implant system of FIG. 1A;

FIG. 1D is a block diagram of the cochlear implant system of FIG. 1A;

FIGS. 2A and 2B are diagrams of illustrating exemplary cochlear implant systems with which aspects of the techniques presented herein can be implemented;

FIGS. 3A and 3B are diagrams of illustrating adjusting operations of the cochlear implant systems of FIGS. 2A and 2B;

FIG. 4 is a table illustrating example parameters of a non-standardized link in various situations;

FIGS. 5A-5F are schematic views of the air efficiency of different example operating modes;

FIG. 6 is a flow diagram of a method of adjusting operations of a first wireless protocol of a cochlear implant according to techniques presented herein;

FIG. 7 is a schematic diagram illustrating an implantable stimulator system with which aspects of the techniques presented herein can be implemented;

FIG. 8 is a schematic diagram illustrating a vestibular stimulator system with which aspects of the techniques presented herein can be implemented; and

FIG. 9 is a schematic diagram illustrating a retinal prosthesis system with which aspects of the techniques presented herein can be implemented.

### DETAILED DESCRIPTION

A device can include at least one wireless interface that operates to transmit and/or receive first wireless data (first data packets) on a first wireless link, and contemporaneously transmit and/or receive second wireless data (second data packets) on a second wireless link. Different wireless protocols, such as Bluetooth®, Bluetooth® Low Energy (BLE), other standardized protocols, and/or non-standardized/proprietary protocols can share the same frequency spectrum. Therefore, when multiple wireless links are transmitting data/packets at the same time, limitations can exist to, for example, airtime and over-air bandwidth. The limitations are often caused by the existence of multiple audio channels and the necessity of retransmissions. Retransmission of data/packets can be needed to overcome packet loss due to radio signal fading and interference from neighbor transceivers operating inside the same frequency band (i.e., 2.4 GHz), which can cause packet collisions. Packets can be retransmitted if the packets have been damaged or lost. In such arrangements, collisions (e.g., due to the same frequency spectrum) can occur that degrade the data (e.g., audio) quality on the first wireless link and/or the second wireless link.

Presented herein are techniques for adjusting one or more parameters or operations associated with a first wireless link operating in accordance with a first wireless protocol (e.g., a non-standardized/proprietary wireless protocol) based on one or more parameters or operations associated with a second wireless link operating in accordance with a second wireless protocol (e.g., a standardized protocol). By adjusting parameters and/or operations associated with the first wireless link (e.g., the link operating in accordance with the non-standardized/proprietary protocol), the contemporaneous operation of both the first wireless link and the second wireless link can be optimized. For example, data (e.g., audio) quality for both links can be improved while minimizing or avoiding dropped packets.

In certain embodiments, the techniques described herein facilitate an in-system radio link with a short duration, such as an incoming call from a smartphone to a behind-the-ear (BTE)/off-the-ear (OTE) device or an implantable device over Bluetooth low energy (BLE) audio, which can temporarily lower the audio quality of another wireless link (e.g., an ipsilateral non-standardized radio link from an external portion to an implantable portion of a hearing device at 2.4 GHz). In particular, embodiments described herein provide for dynamic and automatic changes to one or more operations or parameters of a first wireless link based on one or more operations or parameters a second wireless link. For example, the system can dynamically adjust an audio compression ratio of the codec to change a bit rate of data sent on the first wireless link, a number of retransmissions associated with the first wireless link, etc. to provide the best audio experience versus the lowest over-over bandwidth (airtime) when the second wireless link from an external device is being used.

Merely for ease of description, the techniques presented herein are primarily described with reference to a specific implantable medical device system, namely a cochlear implant system. However, it is to be appreciated that the techniques presented herein can also be partially or fully implemented by other types of implantable medical devices. For example, the techniques presented herein can be implemented by other auditory prosthesis systems that include one or more other types of auditory prostheses, such as middle ear auditory prostheses, bone conduction devices, direct acoustic stimulators, electro-acoustic prostheses, auditory brain stimulators, combinations or variations thereof, etc. The techniques presented herein can also be implemented by dedicated tinnitus therapy devices and tinnitus therapy device systems. In further embodiments, the presented herein can also be implemented by, or used in conjunction with, vestibular devices (e.g., vestibular implants), visual devices (i.e., bionic eyes), sensors, pacemakers, drug delivery systems, defibrillators, functional electrical stimulation devices, catheters, seizure devices (e.g., devices for monitoring and/or treating epileptic events), sleep apnea devices, electroporation devices, etc.

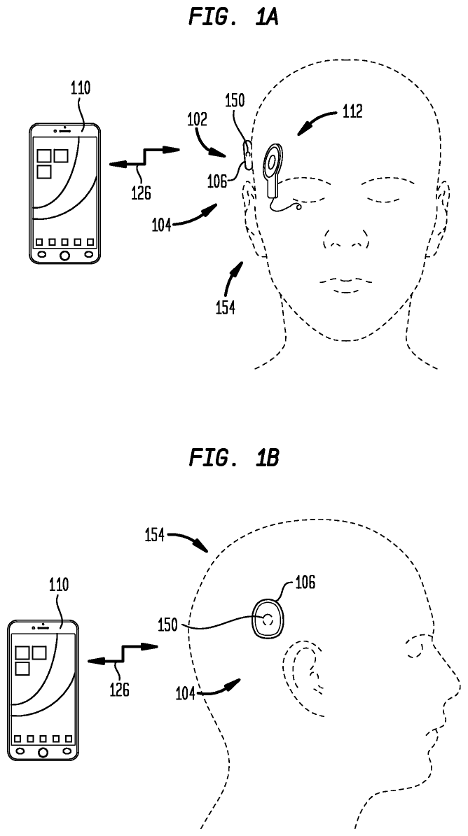

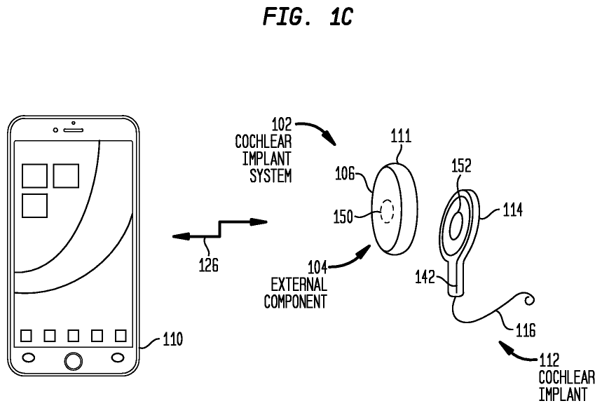

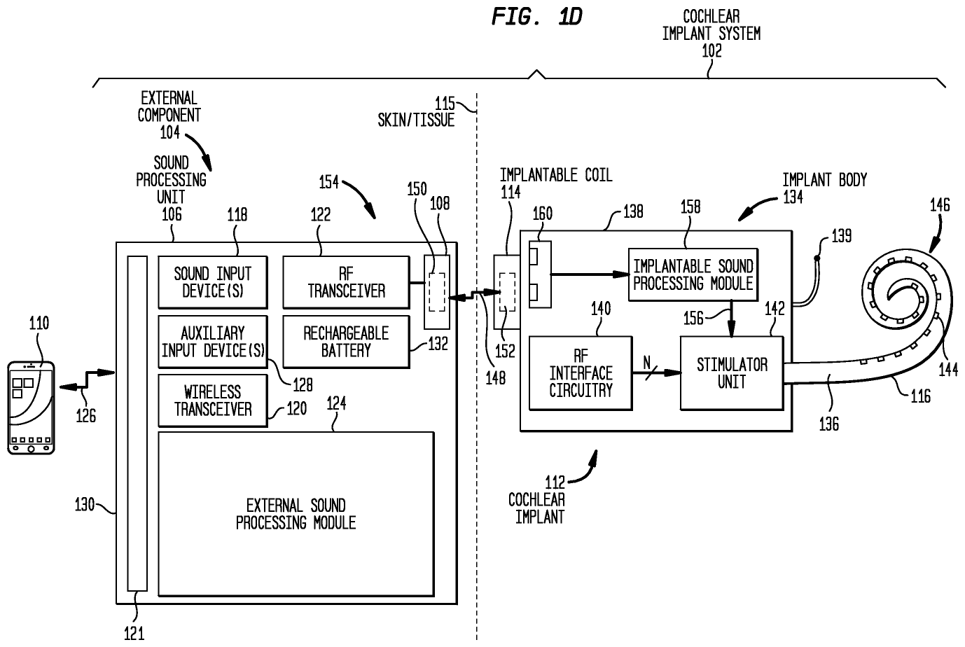

FIGS. 1A-1D illustrates an example cochlear implant system 102 with which aspects of the techniques presented herein can be implemented. The cochlear implant system 102 comprises an external component 104 and an implantable component 112. In the examples of FIGS. 1A-1D, the implantable component is sometimes referred to as a “cochlear implant.” FIG. 1A illustrates the cochlear implant 112 implanted in the head 154 of a recipient, while FIG. 1B is a schematic drawing of the external component 104 worn on the head 154 of the recipient. FIG. 1C is another schematic view of the cochlear implant system 102, while FIG. 1D illustrates further details of the cochlear implant system 102. For ease of description, FIGS. 1A-1D will generally be described together.

Cochlear implant system 102 includes an external component 104 that is configured to be directly or indirectly attached to the body of the recipient and an implantable component 112 configured to be implanted in the recipient. In the examples of FIGS. 1A-1D, the external component 104 comprises a sound processing unit 106, while the cochlear implant 112 includes an implantable coil 114, an implant body 134, and an elongate stimulating assembly 116 configured to be implanted in the recipient's cochlea.

In the example of FIGS. 1A-1D, the sound processing unit 106 is an off-the-ear (OTE) sound processing unit, sometimes referred to herein as an OTE component, that is configured to send data and power to the implantable component 112. In general, an OTE sound processing unit is a component having a generally cylindrically shaped housing 111 and which is configured to be magnetically coupled to the recipient's head (e.g., includes an integrated external magnet 150 configured to be magnetically coupled to an implantable magnet 152 in the implantable component 112). The OTE sound processing unit 106 also includes an integrated external (headpiece) coil 108 that is configured to be inductively coupled to the implantable coil 114.

It is to be appreciated that the OTE sound processing unit 106 is merely illustrative of the external devices that could operate with implantable component 112. For example, in alternative examples, the external component can comprise a behind-the-ear (BTE) sound processing unit or a micro-BTE sound processing unit and a separate external. In general, a BTE sound processing unit comprises a housing that is shaped to be worn on the outer ear of the recipient and is connected to the separate external coil assembly via a cable, where the external coil assembly is configured to be magnetically and inductively coupled to the implantable coil 114. It is also to be appreciated that alternative external components could be located in the recipient's ear canal, worn on the body, etc.

As noted above, the cochlear implant system 102 includes the sound processing unit 106 and the cochlear implant 112. However, as described further below, the cochlear implant 112 can operate independently from the sound processing unit 106, for at least a period, to stimulate the recipient. For example, the cochlear implant 112 can operate in a first general mode, sometimes referred to as an “external hearing mode,” in which the sound processing unit 106 captures sound signals which are then used as the basis for delivering stimulation signals to the recipient. The cochlear implant 112 can also operate in a second general mode, sometimes referred as an “invisible hearing” mode, in which the sound processing unit 106 is unable to provide sound signals to the cochlear implant 112 (e.g., the sound processing unit 106 is not present, the sound processing unit 106 is powered-off, the sound processing unit 106 is malfunctioning, etc.). As such, in the invisible hearing mode, the cochlear implant 112 captures sound signals itself via implantable sound sensors and then uses those sound signals as the basis for delivering stimulation signals to the recipient. Further details regarding operation of the cochlear implant 112 in the external hearing mode are provided below, followed by details regarding operation of the cochlear implant 112 in the invisible hearing mode. It is to be appreciated that reference to the external hearing mode and the invisible hearing mode is merely illustrative and that the cochlear implant 112 could also operate in alternative modes.

In FIGS. 1A and 1C, the cochlear implant system 102 is shown with an external device 110, configured to implement aspects of the techniques presented. The external device 110 is a computing device, such as a computer (e.g., laptop, desktop, tablet), a mobile phone, remote control unit, etc. As described further below, the external device 110 comprises a telephone enhancement module that, as described further below, is configured to implement aspects of the auditory rehabilitation techniques presented herein for independent telephone usage. The external device 110 and the cochlear implant system 102 (e.g., OTE sound processing unit 106 or the cochlear implant 112) wirelessly communicate via a bi-directional communication link 126. The bi-directional communication link 126 can comprise, for example, a short-range communication, such as Bluetooth link, Bluetooth Low Energy (BLE) link, a non-standardized link, etc.

Returning to the example of FIGS. 1A-1D, the OTE sound processing unit 106 comprises one or more input devices that are configured to receive input signals (e.g., sound or data signals). The one or more input devices include one or more sound input devices 118 (e.g., one or more external microphones, audio input ports, telecoils, etc.), one or more auxiliary input devices 128 (e.g., audio ports, such as a Direct Audio Input (DAI), data ports, such as a Universal Serial Bus (USB) port, cable port, etc.), and a wireless transmitter/receiver (transceiver) 120 (e.g., for communication with the external device 110). However, it is to be appreciated that one or more input devices can include additional types of input devices and/or less input devices (e.g., the wireless short range radio transceiver 120 and/or one or more auxiliary input devices 128 could be omitted).

The OTE sound processing unit 106 also comprises the external coil 108, a charging coil 130, a closely-coupled transmitter/receiver (RF transceiver) 122, sometimes referred to as or radio-frequency (RF) transceiver 122, at least one rechargeable battery 132, and an external sound processing module 124. The external sound processing module 124 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

The implantable component 112 comprises an implant body (main module) 134, a lead region 136, and the intra-cochlear stimulating assembly 116, all configured to be implanted under the skin/tissue (tissue) 115 of the recipient. The implant body 134 generally comprises a hermetically-sealed housing 138 in which RF interface circuitry 140 and a stimulator unit 142 are disposed. The implant body 134 also includes the internal/implantable coil 114 that is generally external to the housing 138, but which is connected to the RF interface circuitry 140 via a hermetic feedthrough (not shown in FIG. 1D).

As noted, stimulating assembly 116 is configured to be at least partially implanted in the recipient's cochlea. Stimulating assembly 116 includes a plurality of longitudinally spaced intra-cochlear electrical stimulating contacts (electrodes) 144 that collectively form a contact or electrode array 146 for delivery of electrical stimulation (current) to the recipient's cochlea.

Stimulating assembly 116 extends through an opening in the recipient's cochlea (e.g., cochleostomy, the round window, etc.) and has a proximal end connected to stimulator unit 142 via lead region 136 and a hermetic feedthrough (not shown in FIG. 1D). Lead region 136 includes a plurality of conductors (wires) that electrically couple the electrodes 144 to the stimulator unit 142. The implantable component 112 also includes an electrode outside of the cochlea, sometimes referred to as the extra-cochlear electrode (ECE) 139.

As noted, the cochlear implant system 102 includes the external coil 108 and the implantable coil 114. The external magnet 152 is fixed relative to the external coil 108 and the implantable magnet 152 is fixed relative to the implantable coil 114. The magnets fixed relative to the external coil 108 and the implantable coil 114 facilitate the operational alignment of the external coil 108 with the implantable coil 114. This operational alignment of the coils enables the external component 104 to transmit data and power to the implantable component 112 via a closely-coupled wireless link 148 formed between the external coil 108 with the implantable coil 114. In certain examples, the closely-coupled wireless link 148 is a radio frequency (RF) link. However, various other types of energy transfer, such as infrared (IR), electromagnetic, capacitive and inductive transfer, can be used to transfer the power and/or data from an external component to an implantable component and, as such, FIG. 1D illustrates only one example arrangement. According to techniques described herein, operations of the wireless link 148 can be adjusted when the external component 104 or the implantable component 112 receives data (e.g., sound data or an audio stream) via another wireless link from an external device.

As noted above, sound processing unit 106 includes the external sound processing module 124. The external sound processing module 124 is configured to convert received input signals (received at one or more of the input devices) into output signals for use in stimulating a first ear of a recipient (i.e., the external sound processing module 124 is configured to perform sound processing on input signals received at the sound processing unit 106). Stated differently, the one or more processors in the external sound processing module 124 are configured to execute sound processing logic in memory to convert the received input signals into output signals that represent electrical stimulation for delivery to the recipient.

As noted, FIG. 1D illustrates an embodiment in which the external sound processing module 124 in the sound processing unit 106 generates the output signals. In an alternative embodiment, the sound processing unit 106 can send less processed information (e.g., audio data) to the implantable component 112 and the sound processing operations (e.g., conversion of sounds to output signals) can be performed by a processor within the implantable component 112.

Returning to the specific example of FIG. 1D, the output signals are provided to the RF transceiver 122, which transcutaneously transfers the output signals (e.g., in an encoded manner) to the implantable component 112 via external coil 108 and implantable coil 114. That is, the output signals are received at the RF interface circuitry 140 via implantable coil 114 and provided to the stimulator unit 142. The stimulator unit 142 is configured to utilize the output signals to generate electrical stimulation signals (e.g., current signals) for delivery to the recipient's cochlea. In this way, cochlear implant system 102 electrically stimulates the recipient's auditory nerve cells, bypassing absent or defective hair cells that normally transduce acoustic vibrations into neural activity, in a manner that causes the recipient to perceive one or more components of the received sound signals.

As detailed above, in the external hearing mode the cochlear implant 112 receives processed sound signals from the sound processing unit 106. However, in the invisible hearing mode, the cochlear implant 112 is configured to capture and process sound signals for use in electrically stimulating the recipient's auditory nerve cells. In particular, as shown in FIG. 1D, the cochlear implant 112 includes a plurality of implantable sound sensors 160 and an implantable sound processing module 158. Similar to the external sound processing module 124, the implantable sound processing module 158 can comprise, for example, one or more processors and a memory device (memory) that includes sound processing logic. The memory device can comprise any one or more of: Non-Volatile Memory (NVM), Ferroelectric Random Access Memory (FRAM), read only memory (ROM), random access memory (RAM), magnetic disk storage media devices, optical storage media devices, flash memory devices, electrical, optical, or other physical/tangible memory storage devices. The one or more processors are, for example, microprocessors or microcontrollers that execute instructions for the sound processing logic stored in memory device.

In the invisible hearing mode, the implantable sound sensors 160 are configured to detect/capture signals (e.g., acoustic sound signals, vibrations, etc.), which are provided to the implantable sound processing module 158. The implantable sound processing module 158 is configured to convert received input signals (received at one or more of the implantable sound sensors 160) into output signals for use in stimulating the first ear of a recipient (i.e., the processing module 158 is configured to perform sound processing operations). Stated differently, the one or more processors in implantable sound processing module 158 are configured to execute sound processing logic in memory to convert the received input signals into output signals 156 that are provided to the stimulator unit 142. The stimulator unit 142 is configured to utilize the output signals 156 to generate electrical stimulation signals (e.g., current signals) for delivery to the recipient's cochlea, thereby bypassing the absent or defective hair cells that normally transduce acoustic vibrations into neural activity.

It is to be appreciated that the above description of the so-called external hearing mode and the so-called invisible hearing mode are merely illustrative and that the cochlear implant system 102 could operate differently in different embodiments. For example, in one alternative implementation of the external hearing mode, the cochlear implant 112 could use signals captured by the sound input devices 118 and the implantable sound sensors 160 in generating stimulation signals for delivery to the recipient.

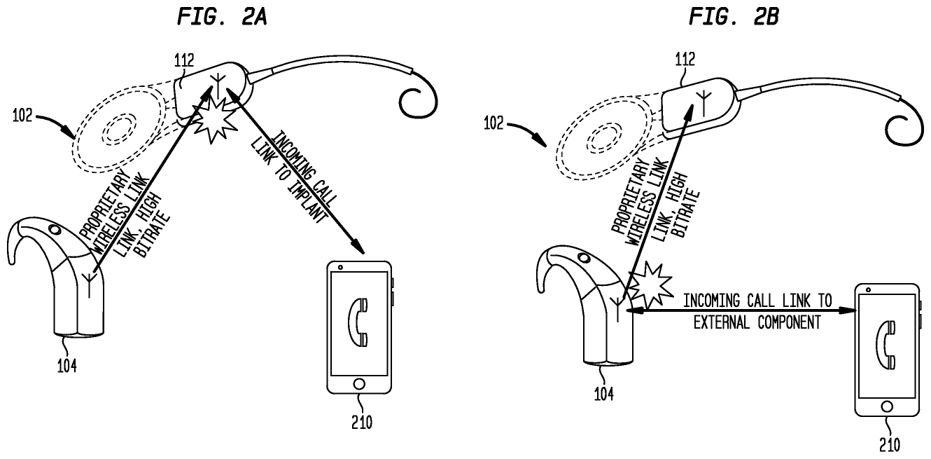

FIGS. 2A and 2B illustrate an example cochlear implant system 102 with which aspects of the techniques presented herein can be implemented.

In the example illustrated in FIGS. 2A and 2B, cochlear implant system 102 includes external component 104, cochlear implant 112, and an external device, such as a user device 210. User device 210 can be, for example, a cellular telephone, a tablet, an audio player, or another device capable of forming a wireless connection with external component 104 and/or cochlear implant 112 over a communication link that can comprise, for example, a short-range communication, such as a Bluetooth® link. In the example illustrated in FIGS. 2A and 2B, user device 210 is a cellular telephone.

As illustrated in FIG. 2A, external component 104 has established a non-standardized/proprietary wireless link with cochlear implant 112. For example, external component 104 can transmit data to cochlear implant 112 using a transmission protocol with a relatively high bitrate. User device 210 can receive a phone call and can establish a wireless link with cochlear implant 112 to transmit audio data from the phone call. In this example, cochlear implant 112 has established a first link with external component 104 using a first wireless protocol and a second link with user device 210 using a second wireless protocol. In this example, packets transmitted on the relatively high bitrate link between external component 104 and cochlear implant 112 collide with packets on the incoming link between user device 210 and cochlear implant 112. When a lot of collisions occur, the quality of one or both of the links is degraded.

In the example illustrated in FIG. 2B, external component 104 has established a relatively high bitrate non-standardized wireless link with cochlear implant 112 and user device 210 has established a wireless link with external component 104 to transmit audio data in response to receiving a phone call. In this example, external component 104 has established a first link with cochlear implant 112 using a first wireless protocol and a second link with user device 210 using a second wireless protocol. Since both wireless protocols operate inside the same frequency band with limited bandwidth, packets transmitted on the relatively high bitrate link between external component 104 and cochlear implant 112 collide with packets transmitted on the incoming link from user device 210 to external component 104. In this example, the collisions can result in a degraded audio quality of one or both of the links.

In the examples described above in FIGS. 2A and 2B, parameters and/or operations of the wireless link (e.g., the wireless protocol associated with the non-standardized link) between external component 104 and cochlear implant 112 can be adjusted to improve the audio quality of one or both of the links. As further described below, the parameters and/or operations of the wireless protocol associated with the non-standardized link can be dynamically adjusted by adjusting the audio compression ratio of the codec and/or the number of retransmissions on the ipsilateral non-standardized channel.

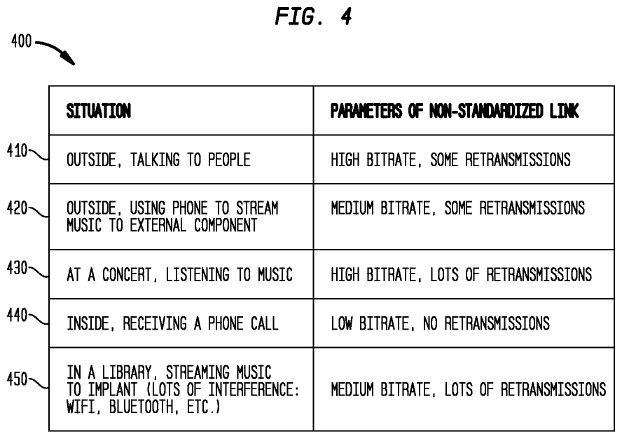

As further described below with respect to FIG. 4, a manner in which the parameters and/or the operations are adjusted can depend on a number of different factors. These factors can include, for example, a required number of in-system radio streams, a current auditory environment (e.g., opera, speech, conversation, music concert, restaurant, meeting, etc.), an indoor location vs. an outdoor location (noise), a current activity of a recipient of the hearing device (e.g., walking, running, playing sports, etc.), a human voice in the transmitted data (e.g., male, female, child), a current means of transportation, etc.

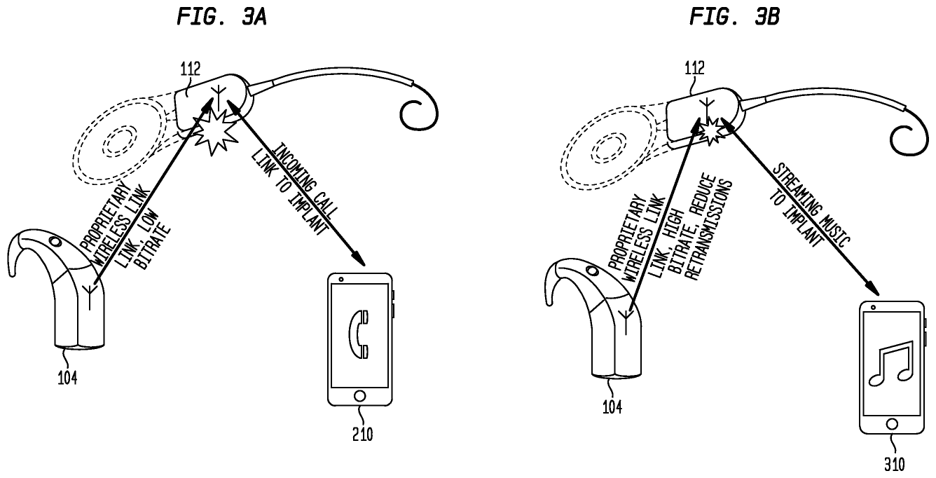

FIGS. 3A and 3B illustrate example cochlear implant systems 102 with which aspects of the techniques presented herein can be implemented.

FIG. 3A illustrates the example described above with respect to FIG. 2A in which the non-standardized link has been established between external component 104 and cochlear implant 112 using a first transmission protocol and a second link has been established between user device 210 and cochlear implant 112 using a second transmission protocol (e.g., over a Bluetooth link). In the example illustrated in FIG. 3A, the codec of the non-standardized link is adjusted so the bitrate is reduced such that the data is transmitted between external component 104 and cochlear implant 112 using a lower/reduced bitrate (e.g., a bit rate than is lower than typically used at the non-standardized link). When the bitrate is reduced, fewer and/or shorter packets are transmitted on the non-standardized link, which reduces the number of collisions between packets transmitted on the non-standardized link and packets transmitted on the link between user device 210 and cochlear implant 112. By reducing the bitrate, the throughput of the non-standardized link is reduced. However, with fewer collisions, both data streams are more reliable and arrive with packet loss at cochlear implant 112.

FIG. 3B illustrates an example similar to the example described above with respect to FIG. 2B. In this example, external component 104 has established a relatively high bitrate link with cochlear implant 112 and a user device 310 has established a second link with cochlear implant 112. In this example, user device 310 is streaming music to cochlear implant 112.

In the example illustrated in FIG. 3B, to reduce collisions between the two links, the non-standardized link maintains a relatively high bitrate, but the number of retransmissions is reduced. In other words, external component 104 reduces the rate of retransmitting packets to cochlear implant 112 (e.g., when the packets have been damaged or lost). By reducing the retransmission rate, the number of packets transmitted to cochlear implant 112 is decreased, which causes fewer collisions. Although reducing the retransmission rate can lead to a temporarily less reliable non-standardized link, both data streams arrive at cochlear implant 112 with fewer collisions.

FIG. 4 illustrates a table 400 showing examples in which the operations/parameters of the non-standardized link are adjusted in different situations. As discussed above, operations/parameters of the non-standardized link can be adjusted in different ways to provide the best audio experience in different scenarios/conditions. A current audio environment as well as additional factors, such as a type of audio associated with the second link, whether external component 104 or cochlear implant 112 is receiving the audio on the second link, a number of other radio links in the vicinity of the recipient of the hearing device, etc., can affect how the operations/parameters of the non-standardized link are adjusted. A determination of how to adjust the operations/parameters can be made at external component 104, cochlear implant 112, or at another device such as external device 110.

In the examples shown in table 400, the parameters/operations of the non-standardized link can be adjusted based on a current situation of a user even when a second link has not been established. As illustrated at entry 410, when a user is outside talking to people, the non-standardized link can maintain a relatively high bitrate, but the retransmission rate can be lowered slightly to allow some retransmissions. As illustrated at 420, when the user is outside using a phone to stream music to external component 104, the bitrate of the non-standardized link can be lowered to “medium” and the retransmission rate can also be lowered to allow some retransmissions.

In the example shown in entry 430, when a user is at a concert listening to music, the non-standardized link can have a relatively high bitrate with lots of retransmission to provide the highest quality and most reliable audio experience. In the example shown at entry 440, when the user is inside and receives a phone call, the user can be focused on the phone call received via the second link, so the parameters of the non-standardized link can be adjusted to provide a low bitrate with no retransmissions. In this way, the audio quality of the phone call can be maximized. In the example shown at entry 450, when the user is in a library streaming music to cochlear implant 112 (e.g., with lots of interference from other devices'Wi-Fi and Bluetooth connections), the non-standardized link can have a medium bitrate with lots of retransmission.

The entries shown in table 400 are exemplary and the parameters can be adjusted in different or additional ways. Furthermore, although only five examples are given in table 400, the operations/parameters of the non-standardized link can be adjusted in many other ways in different situations.

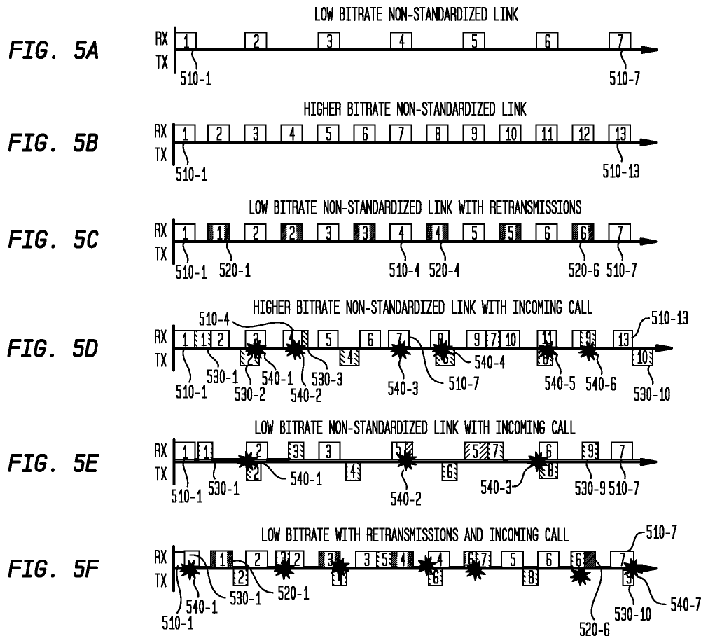

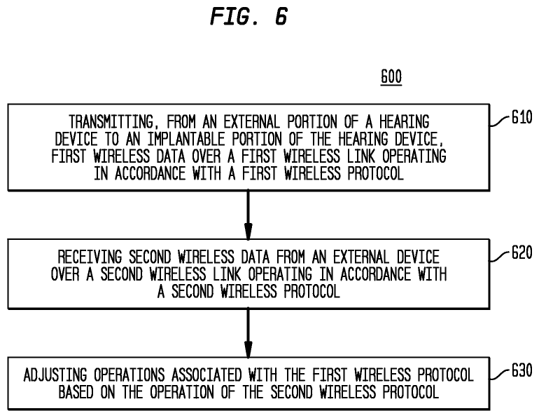

FIGS. 5A-5E illustrate exemplary schematic views of air efficiency of different modes of transmission of the non-standardized link. As described above with respect to FIG. 4, the operation of the non-standardized link can switch between different modes based on different situations or other factors. FIGS. 5A-5E illustrate packets of data and collisions between packets. In some situations, only some of the data packets and/or collisions are labeled for simplicity.

FIG. 5A illustrates the air efficiency of a low bitrate non-standardized link and FIG. 5B illustrates the air efficiency of a higher bitrate non-standardized link. The receiver antenna (Rx) of the low bitrate non-standardized link illustrated in FIG. 5A receives seven (7) packets 510-1 to 510-7 over a first link in a period of time and the receiver of the higher bitrate non-standardized link illustrated in FIG. 5B receives 13 packets over the first link in the period of time. In FIGS. 5A and 5B, only the first packets (510-1) and the final packets (510-7 in FIG. 5A and 510-13 in FIG. 5B) are labeled for simplicity. The higher bitrate non-standardized link receives packets at a greater rate than the lower bitrate non-standardized link.

FIG. 5C illustrates the air efficiency of a low bitrate non-standardized link with retransmissions. As illustrated in FIG. 5C, the receiver receives packets 510-1 to 510-7 and retransmitted packets 520-1 to 520-6. For example, the receiver receives packet 510-1 and retransmitted packet 520-1. As another example, Rx receives packet 510-4 and retransmitted packet 520-4. Packet 510-1 and retransmitted packet 520-1 can include the same data or payload (and, in the same manner, packet 510-4 and retransmitted packet 520-4 can include the same data or payload). Retransmitted packets 520-1 to 520-6 can be transmitted, for example, when packets 510-1 to 510-6 are lost or damaged.

FIG. 5D illustrates the air efficiency of a relatively high bitrate non-standardized link using a first wireless protocol when there is an incoming call over a second link to external component 104 or cochlear implant 112 using a second wireless protocol (e.g., using Bluetooth). In the example illustrated in FIG. 5D, the receiver receives packets 510-1 to 510-13 over the non-standardized link. In addition, a transmitter antenna (Tx) transmits additional packets 530-1 to 530-10 over the second link. Because the non-standardized link has a relatively high bitrate, packets 510-1 to 510-13 are being transmitted/received at a high rate, which leads to collisions with packets transmitted/received on the second link.

As illustrated in FIG. 5D, the receiver receives packet 530-1 over the second link, but there is a collision 540-1 when packet 530-2 is transmitted over the second link. Another collision 540-2 occurs when packet 510-4 is received over the non-standardized link and packet 530-3 is received over the second link. Collision 540-3 occurs when packet 510-7 is received over the non-standardized link. Collisions 540-4 to 540-6 additionally occur when packets being transmitted over the non-standardized link and the second link collide. As shown in FIG. 5D, multiple collisions occur when a phone call is received over a second link when the non-standardized link has a relatively high bitrate, leading to a loss of packets. This can result in a low quality for audio received over the non-standardized link and/or the second link.

FIG. 5E illustrates the air efficiency of a low bitrate non-standardized link using a first wireless protocol when there is an incoming call over a second link to external component 104 or cochlear implant 112 using a second wireless protocol (e.g., using Bluetooth). As illustrated in FIG. 5E, packets 510-1 to 510-7 are transmitted over the non-standardized link. In addition, packets 530-1 to 530-9 are transmitted over the second link. Because the non-standardized link has a low bitrate and fewer packets are transmitted over the non-standardized link during a time period compared to the higher bitrate example described with respect to FIG. 5D, three (3) collisions 540-1 to 540-3 occur (instead of six (6) collisions when the non-standardized link has a higher bitrate).

FIG. 5F illustrates the air efficiency of a low bitrate non-standardized link using a first wireless protocol when there is an incoming call over a second link to external component 104 or cochlear implant 112 using a second wireless protocol (e.g., via Bluetooth) and when packets are retransmitted on the non-standardized link. As illustrated in FIG. 5F, packets 510-1 to 510-7 and retransmitted packets 520-1 to 520-6 are transmitted on the non-standardized link and packets 530-1 to 530-10 are transmitted on the second link, which results in six (6) collisions 540-1 to 540-6. In this example, although a lot of collisions occur, all audio data is still received on the non-standardized link because of the retransmissions. Therefore, the user experiences a great audio quality on the non-standardized link when packets are retransmitted.

FIG. 6 is a flow chart of a method 600 of adjusting operations associated with the first wireless protocol (adjusting a first wireless link) in response to receiving data via a second wireless link, according to embodiments herein. In particular, at 610, first wireless data is transmitted from an external portion of a hearing device to an implantable portion of the hearing device over a first wireless link operating in accordance with a first wireless protocol. For example, external component 104 can transmit an audio stream to cochlear implant 112. At 620, second wireless data is received from an external device over a second wireless link operating in accordance with a second wireless protocol. For example, external component 104 or cochlear implant 112 can receive a second audio stream from an external device using the second wireless protocol. At 630, operations associated with the first wireless protocol are adjusted based on the operation of the second wireless protocol. For example, a compression ratio of a codec can be adjusted to change a bitrate associated with the first wireless link and/or a number of retransmissions associated with the first wireless link can be adjusted.

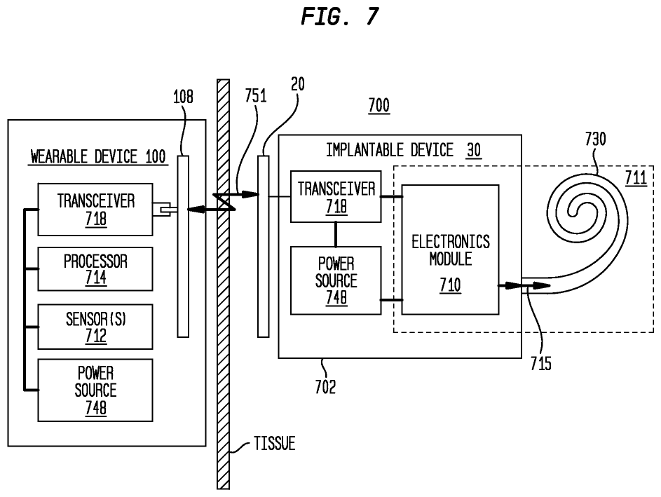

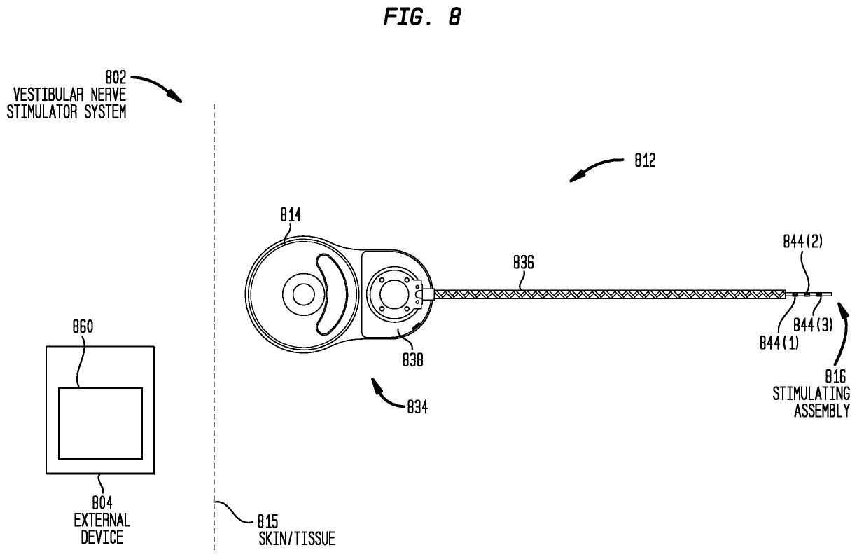

As previously described, the technology disclosed herein can be applied in any of a variety of circumstances and with a variety of different devices. Example devices that can benefit from technology disclosed herein are described in more detail in FIGS. 7-9, below. As described below, the operating parameters for the devices described with reference to FIGS. 7-9 can be adjusted using methods described above with respect to FIG. 8. For example, the techniques described herein can be used to adjust operating parameters of wearable medical devices, such as an implantable stimulation system as described in FIG. 7, a vestibular stimulator as described in FIG. 8, or a retinal prosthesis as described in FIG. 7. The techniques of the present disclosure can be applied to other medical devices, such as neurostimulators, cardiac pacemakers, cardiac defibrillators, sleep apnea management stimulators, seizure therapy stimulators, tinnitus management stimulators, and vestibular stimulation devices, as well as other medical devices that deliver stimulation to tissue. Further, technology described herein can also be applied to consumer devices. These different systems and devices can benefit from the technology described herein.

FIG. 7 is a functional block diagram of an implantable stimulator system 700 that can benefit from the technologies described herein. The implantable stimulator system 700 includes the wearable device 100 acting as an external processor device and an implantable device 30 acting as an implanted stimulator device. In examples, the implantable device 30 is an implantable stimulator device configured to be implanted beneath a recipient's tissue (e.g., skin). In examples, the implantable device 30 includes a biocompatible implantable housing 702. Here, the wearable device 100 is configured to transcutaneously couple with the implantable device 30 via a wireless connection to provide additional functionality to the implantable device 30.

In the illustrated example, the wearable device 100 includes one or more sensors 712, a processor 714, a transceiver 718, and a power source 748. The one or more sensors 712 can be one or more units configured to produce data based on sensed activities. In an example where the stimulation system 700 is an auditory prosthesis system, the one or more sensors 712 include sound input sensors, such as a microphone, an electrical input for an FM hearing system, other components for receiving sound input, or combinations thereof. Where the stimulation system 700 is a visual prosthesis system, the one or more sensors 712 can include one or more cameras or other visual sensors. Where the stimulation system 700 is a cardiac stimulator, the one or more sensors 712 can include cardiac monitors. The processor 714 can be a component (e.g., a central processing unit) configured to control stimulation provided by the implantable device 30. The stimulation can be controlled based on data from the sensor 712, a stimulation schedule, or other data. Where the stimulation system 700 is an auditory prosthesis, the processor 714 can be configured to convert sound signals received from the sensor(s) 712 (e.g., acting as a sound input unit) into signals 751. The transceiver 718 is configured to send the signals 751 in the form of power signals, data signals, combinations thereof (e.g., by interleaving the signals), or other signals. The transceiver 718 can also be configured to receive power or data. Stimulation signals can be generated by the processor 714 and transmitted, using the transceiver 718, to the implantable device 30 for use in providing stimulation.

In the illustrated example, the implantable device 30 includes a transceiver 718, a power source 748, and a medical instrument 711 that includes an electronics module 710 and a stimulator assembly 730. The implantable device 30 further includes a hermetically sealed, biocompatible implantable housing 702 enclosing one or more of the components.

The electronics module 710 can include one or more other components to provide medical device functionality. In many examples, the electronics module 710 includes one or more components for receiving a signal and converting the signal into the stimulation signal 715. The electronics module 710 can further include a stimulator unit. The electronics module 710 can generate or control delivery of the stimulation signals 715 to the stimulator assembly 730. In examples, the electronics module 710 includes one or more processors (e.g., central processing units or microcontrollers) coupled to memory components (e.g., flash memory) storing instructions that when executed cause performance of an operation. In examples, the electronics module 710 generates and monitors parameters associated with generating and delivering the stimulus (e.g., output voltage, output current, or line impedance). In examples, the electronics module 710 generates a telemetry signal (e.g., a data signal) that includes telemetry data. The electronics module 710 can send the telemetry signal to the wearable device 100 or store the telemetry signal in memory for later use or retrieval.

The stimulator assembly 730 can be a component configured to provide stimulation to target tissue. In the illustrated example, the stimulator assembly 730 is an electrode assembly that includes an array of electrode contacts disposed on a lead. The lead can be disposed proximate tissue to be stimulated. Where the system 700 is a cochlear implant system, the stimulator assembly 730 can be inserted into the recipient's cochlea. The stimulator assembly 730 can be configured to deliver stimulation signals 715 (e.g., electrical stimulation signals) generated by the electronics module 710 to the cochlea to cause the recipient to experience a hearing percept. In other examples, the stimulator assembly 730 is a vibratory actuator disposed inside or outside of a housing of the implantable device 30 and configured to generate vibrations. The vibratory actuator receives the stimulation signals 715 and, based thereon, generates a mechanical output force in the form of vibrations. The actuator can deliver the vibrations to the skull of the recipient in a manner that produces motion or vibration of the recipient's skull, thereby causing a hearing percept by activating the hair cells in the recipient's cochlea via cochlea fluid motion.

The transceivers 718 can be components configured to transcutaneously receive and/or transmit a signal 751 (e.g., a power signal and/or a data signal). The transceiver 718 can be a collection of one or more components that form part of a transcutaneous energy or data transfer system to transfer the signal 751 between the wearable device 100 and the implantable device 30. Various types of signal transfer, such as electromagnetic, capacitive, and inductive transfer, can be used to usably receive or transmit the signal 751. The transceiver 718 can include or be electrically connected to a coil 20.

As illustrated, the wearable device 100 includes a coil 108 for transcutaneous transfer of signals with the concave coil 20. As noted above, the transcutaneous transfer of signals between coil 108 and the coil 20 can include the transfer of power and/or data from the coil 108 to the coil 20 and/or the transfer of data from coil 20 to the coil 108. The power source 748 can be one or more components configured to provide operational power to other components. The power source 748 can be or include one or more rechargeable batteries. Power for the batteries can be received from a source and stored in the battery. The power can then be distributed to the other components as needed for operation.

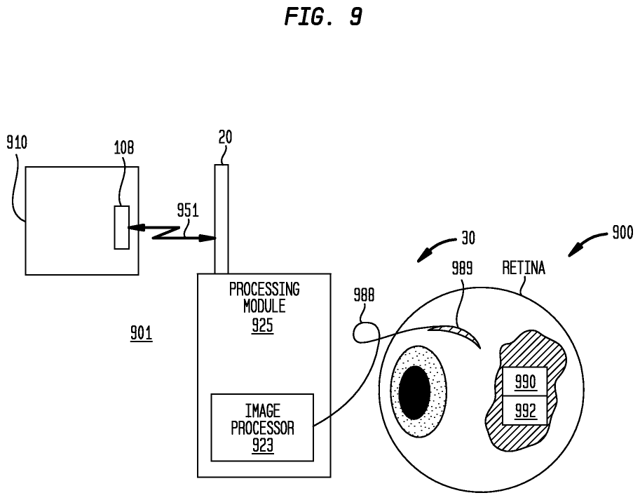

As should be appreciated, while particular components are described in conjunction with FIG. 9, technology disclosed herein can be applied in any of a variety of circumstances. The above discussion is not meant to suggest that the disclosed techniques are only suitable for implementation within systems akin to that illustrated in and described with respect to FIG. 7. In general, additional configurations can be used to practice the methods and systems herein and/or some aspects described can be excluded without departing from the methods and systems disclosed herein.

FIG. 8 illustrates an example vestibular stimulator system 802, with which embodiments presented herein can be implemented. As shown, the vestibular stimulator system 802 comprises an implantable component (vestibular stimulator) 812 and an external device/component 804 (e.g., external processing device, battery charger, remote control, etc.). The external device 804 comprises a transceiver unit 860. As such, the external device 804 is configured to transfer data (and potentially power) to the vestibular stimulator 812,

The vestibular stimulator 812 comprises an implant body (main module) 834, a lead region 836, and a stimulating assembly 816, all configured to be implanted under the skin/tissue (tissue) 815 of the recipient. The implant body 834 generally comprises a hermetically-sealed housing 838 in which RF interface circuitry, one or more rechargeable batteries, one or more processors, and a stimulator unit are disposed. The implant body 134 also includes an internal/implantable coil 814 that is generally external to the housing 838, but which is connected to the transceiver via a hermetic feedthrough (not shown).

The stimulating assembly 816 comprises a plurality of electrodes 844(1)-(3) disposed in a carrier member (e.g., a flexible silicone body). In this specific example, the stimulating assembly 816 comprises three (3) stimulation electrodes, referred to as stimulation electrodes 844(1), 844(2), and 844(3). The stimulation electrodes 844(1), 844(2), and 844(3) function as an electrical interface for delivery of electrical stimulation signals to the recipient's vestibular system.

The stimulating assembly 816 is configured such that a surgeon can implant the stimulating assembly adjacent the recipient's otolith organs via, for example, the recipient's oval window. It is to be appreciated that this specific embodiment with three stimulation electrodes is merely illustrative and that the techniques presented herein can be used with stimulating assemblies having different numbers of stimulation electrodes, stimulating assemblies having different lengths, etc.

In operation, the vestibular stimulator 812, the external device 804, and/or another external device, can be configured to implement the techniques presented herein. That is, the vestibular stimulator 812, possibly in combination with the external device 804 and/or another external device, can include an evoked biological response analysis system, as described elsewhere herein.

FIG. 9 illustrates a retinal prosthesis system 901 that comprises an external device 910 (which can correspond to the wearable device 100) configured to communicate with a retinal prosthesis 900 via signals 951. The retinal prosthesis 900 comprises an implanted processing module 925 (e.g., which can correspond to the implantable device 30) and a retinal prosthesis sensor-stimulator 990 is positioned proximate the retina of a recipient. The external device 910 and the processing module 925 can communicate via coils 108, 20.

In an example, sensory inputs (e.g., photons entering the eye) are absorbed by a microelectronic array of the sensor-stimulator 990 that is hybridized to a glass piece 992 including, for example, an embedded array of microwires. The glass can have a curved surface that conforms to the inner radius of the retina. The sensor-stimulator 990 can include a microelectronic imaging device that can be made of thin silicon containing integrated circuitry that convert the incident photons to an electronic charge.

The processing module 925 includes an image processor 923 that is in signal communication with the sensor-stimulator 990 via, for example, a lead 988 which extends through surgical incision 989 formed in the eye wall. In other examples, processing module 925 is in wireless communication with the sensor-stimulator 990. The image processor 923 processes the input into the sensor-stimulator 990, and provides control signals back to the sensor-stimulator 990 so the device can provide an output to the optic nerve. That said, in an alternate example, the processing is executed by a component proximate to, or integrated with, the sensor-stimulator 990. The electric charge resulting from the conversion of the incident photons is converted to a proportional amount of electronic current which is input to a nearby retinal cell layer. The cells fire and a signal is sent to the optic nerve, thus inducing a sight perception.

The processing module 925 can be implanted in the recipient and function by communicating with the external device 910, such as a behind-the-ear unit, a pair of eyeglasses, etc. The external device 910 can include an external light/image capture device (e.g., located in/on a behind-the-ear device or a pair of glasses, etc.), while, as noted above, in some examples, the sensor-stimulator 990 captures light/images, which sensor-stimulator is implanted in the recipient.

As should be appreciated, while particular uses of the technology have been illustrated and discussed above, the disclosed technology can be used with a variety of devices in accordance with many examples of the technology. The above discussion is not meant to suggest that the disclosed technology is only suitable for implementation within systems akin to that illustrated in the figures. In general, additional configurations can be used to practice the processes and systems herein and/or some aspects described can be excluded without departing from the processes and systems disclosed herein.

This disclosure described some aspects of the present technology with reference to the accompanying drawings, in which only some of the possible aspects were shown. Other aspects can, however, be embodied in many different forms and should not be construed as limited to the aspects set forth herein. Rather, these aspects were provided so that this disclosure was thorough and complete and fully conveyed the scope of the possible aspects to those skilled in the art.

As should be appreciated, the various aspects (e.g., portions, components, etc.) described with respect to the figures herein are not intended to limit the systems and processes to the particular aspects described. Accordingly, additional configurations can be used to practice the methods and systems herein and/or some aspects described can be excluded without departing from the methods and systems disclosed herein.

According to certain aspects, systems and non-transitory computer readable storage media are provided. The systems are configured with hardware configured to execute operations analogous to the methods of the present disclosure. The one or more non-transitory computer readable storage media comprise instructions that, when executed by one or more processors, cause the one or more processors to execute operations analogous to the methods of the present disclosure.

Similarly, where steps of a process are disclosed, those steps are described for purposes of illustrating the present methods and systems and are not intended to limit the disclosure to a particular sequence of steps. For example, the steps can be performed in differing order, two or more steps can be performed concurrently, additional steps can be performed, and disclosed steps can be excluded without departing from the present disclosure. Further, the disclosed processes can be repeated.

Although specific aspects were described herein, the scope of the technology is not limited to those specific aspects. One skilled in the art will recognize other aspects or improvements that are within the scope of the present technology. Therefore, the specific structure, acts, or media are disclosed only as illustrative aspects. The scope of the technology is defined by the following claims and any equivalents therein.

It is also to be appreciated that the embodiments presented herein are not mutually exclusive and that the various embodiments can be combined with another in any of a number of different manners.

1. A method, comprising:
transmitting, from an external portion of a medical device to an implantable portion of the medical device, first wireless data over a first wireless link operating in accordance with a first wireless protocol;
receiving second wireless data from an external device over a second wireless link operating in accordance with a second wireless protocol; and
adjusting operations associated with the first wireless protocol based on the operation of the second wireless protocol.

2. The method of claim 1, wherein adjusting the operations comprises:
adjusting a compression rate of a codec associated with the first wireless protocol.

3. The method of claim 1, wherein adjusting the operations comprises:
adjusting a number of retransmissions associated with the first wireless protocol.

4. The method of claim 1, wherein adjusting the operations includes:
adjusting a compression ratio of a codec and a number of retransmissions associated with the first wireless protocol.

5. The method of claim 1, wherein the first wireless protocol is a non-standardized protocol associated with the medical device.

6. The method of claim 1, wherein the second wireless protocol is at least one of Bluetooth® or Bluetooth® Low Energy (BLE).

7. The method of claim 1, wherein the second wireless data is received at an external portion of the medical device.

8. The method of claim 1, wherein the second wireless data is received at the implantable portion of the medical device.

9. The method of claim 1, wherein adjusting the operations further comprises: adjusting operations associated with the first wireless protocol based on an acoustic environment associated with the medical device.

10. The method of claim 1, wherein adjusting the operations further comprises:
adjusting operations associated with the first wireless protocol based on a type of audio associated with the second wireless data.

11. The method of claim 1, wherein adjusting the operations further comprises:
adjusting operations associated with the first wireless protocol based on an amount of radio interference associated with the medical device.

12. The method of claim 1, wherein the medical device comprises a single wireless interface that is shared by a first wireless link operating in accordance with the first wireless protocol, and a second wireless link operating in accordance with the second wireless protocol.

13. One or more non-transitory computer readable storage media comprising instructions that, when executed by a processor, cause the processor to:
transmit first data packets from an external portion of a medical device to an implantable portion of the medical device a first wireless link;
receive second data packets from an external device over a second wireless link; and
adjust one or more operating parameters of the first wireless link based on one or more operating parameters associated with the second wireless link.

14. The one or more non-transitory computer readable storage media of claim 13, wherein, when adjusting the one or more operating parameters of the first wireless link, the instructions cause the processor to adjust a compression rate of a codec associated with operation of the first wireless link.

15. The one or more non-transitory computer readable storage media of claim 13, wherein, when adjusting the one or more operating parameters of the first wireless link, the instructions cause the processor to adjust a number of retransmissions associated with operation of the first wireless link.

16. The one or more non-transitory computer readable storage media of claim 13, wherein, when adjusting the one or more operating parameters of the first wireless link, the instructions cause the processor to adjust a compression ratio of a codec and a number of retransmissions associated with operation of the first wireless link.

17. (canceled)

18. (canceled)

19. (canceled)

20. The one or more non-transitory computer readable storage media of claim 13, wherein the second data packets are received at an external portion of the medical device.

21. The one or more non-transitory computer readable storage media of claim 13, wherein the second data packets are received at the implantable portion of the medical device.

22. (canceled)

23. (canceled)

24. The one or more non-transitory computer readable storage media of claim 13, wherein, when adjusting the one or more operating parameters of the first wireless link, the instructions cause the processor to adjust the one or more operating parameters of the first wireless link based on a type of data associated with the second data packets.

25. The one or more non-transitory computer readable storage media of claim 24, wherein the second data packets are audio data packets, and wherein when adjusting the one or more operating parameters of the first wireless link based on a type of data associated with the second data packets, the instructions cause the processor to adjust the one or more operating parameters of the first wireless link based on a type of audio data associated with the second data packets.

26. (canceled)

27. (canceled)

28. (canceled)

29. A device, comprising:
at least one wireless interface;
a memory; and
at least one processor configured to:
transmit, via the at least one wireless interface, first wireless data in accordance with a first wireless protocol,
receive, via the at least one wireless interface, second wireless data sent in accordance with a second wireless protocol, and
adjust one or more parameters of the first wireless protocol data based on one or more parameters of the second wireless protocol.

30. The device of claim 29, wherein, when adjusting the one or more parameters, the at least one processor is configured to adjust a compression rate of a codec associated with the first wireless protocol.

31. The device of claim 29, wherein, when adjusting the one or more parameters, the at least one processor is configured to adjust a number of retransmissions associated with the first wireless protocol.

32. The device of claim 29, wherein, when adjusting the one or more parameters, the at least one processor is configured to adjust a compression ratio of a codec and a number of retransmissions associated with the first wireless protocol.

33. (canceled)

34. (canceled)

35. The device of claim 29, wherein the device is an external component of a medical device system.

36. The device of claim 29, wherein the device is an implantable component of a medical device system.

37. The device of claim 29, wherein the at least one processor is configured to adjust the one or more parameters based on an ambient environment of the device.

38. The device of claim 37, wherein the at least one processor is configured to adjust the one or more parameters based on an ambient acoustic environment of the device.

39. (canceled)

40. (canceled)

41. (canceled)

42. The device of claim 39, wherein the at least one wireless interface comprises a single wireless interface that is shared by a first wireless link operating in accordance with the first wireless protocol, and a second wireless link operating in accordance with the second wireless protocol.
